(19) United States
(12) Patent Application Publication
(10) Pub. No.: US 2025/0262377 A1
(43) Pub. Date: Aug. 21, 2025

(54) MEDICAL DEVICE AND MEDICAL SYSTEM FOR FLUID CONVEYANCE

(71) Applicant: SHENZHEN SISENSING CO., LTD., Shenzhen (CN)

(72) Inventors: Bin XIA, Shenzhen (CN); Yunfeng LI, Shenzhen (CN); Mingsong HAN, Shenzhen (CN); Zhi CHEN, Shenzhen (CN); Junfei FANG, Shenzhen (CN); Ming LAI, Shenzhen (CN)

(73) Assignee: SHENZHEN SISENSING CO., LTD., Shenzhen (CN)

(21) Appl. No.: 18/857,530

(22) PCT Filed: Jul. 15, 2022
(86) PCT No.: PCT/CN2022/105855

§ 371 Date: Oct. 17, 2024

(30) Foreign Application Priority Data
Apr. 26, 2022 (CN) 202210444443.4

(51) Int. Cl. A61M 5/168; A61M 5/172

(52) U.S. Cl. CPC A61M 5/16813; A61M 5/16881; A61M 5/1723; A61M 2205/18; A61M 2205/3576

(57) ABSTRACT

Some embodiments of the disclosure provide medical devices and medical systems for fluid conveyance. In some examples, the medical device includes a reservoir, a first passage, a flow feeder, a driving mechanism, a second passage, and a flow-limiting valve. The flow-limiting valve has a first state and a second state. When the flow-limiting valve is in the first state, the driving mechanism maintains the flow feeder at a first volume, and the flow feeder receives a predetermined volume of fluid from the reservoir via the first passage. When the flow-limiting valve is in the second state, the driving mechanism maintains the flow feeder at a second volume, and the flow feeder provides a predetermined volume of fluid via the second passage. The second volume is smaller than the first volume and the predetermined volume is determined by a volume difference between the first volume and the second volume.

### CROSS REFERENCE TO RELATED APPLICATIONS

This application is the United State national stage entry under 37 U.S.C.. 371 of PCT/CN2022/105855, filed on Jul. 15, 2022, which claims priority to Chinese application number 202210444443.4, filed on Apr. 26, 2022, the disclosure of which are incorporated by reference herein in their entireties.

### FIELD OF THE DISCLOSURE

The disclosure relates generally to the field of medical devices. More specifically, the disclosure relates to medical devices and medical systems for fluid conveyance.

### BACKGROUND

For many chronic diseases, corresponding complications often arise, e.g., chronic diabetes causing complications related to blood glucose. In order to delay or reduce the rapid or persistent effects of chronic diseases on patients, techniques for delivering drugs by automatic injection may be employed on the patients. In current drug delivery technology, the portable drug delivery system is widely used, which usually requires subcutaneous implantation of one tube, when the abnormality of a patient's physiological characteristics reaches early warning, the patient can input the dose of the injected drug through the controller and connect the drug pump filled with medical solution to the previously reserved tube for drug delivery.

However, with the above-mentioned conventional drug delivery techniques, there is still a great difficulty in accurate delivery. If the delivery quantity is too small or too large, there may be a delay in the response time of the drug or an error in the quantity of the drug used, thus resulting in poor treatment effect or medication error. Accordingly, there is an urgent need for a medical device that conveys fluids with high precision.

### SUMMARY

The following presents a simplified summary of the invention in order to provide a basic understanding of some aspects of the invention. This summary is not an extensive overview of the invention. It is not intended to identify critical elements or to delineate the scope of the invention. Its sole purpose is to present some concepts of the invention in a simplified form as a prelude to the more detailed description that is presented elsewhere.

In some embodiments, the first aspect of the disclosure provides a medical device for fluid conveyance, including: a reservoir for containing a fluid, a first passage in communication with the reservoir, a flow feeder for receiving the fluid from the reservoir via the first passage and providing the fluid, a driving mechanism for controlling a volume change of the flow feeder, a second passage in communication with the flow feeder, receiving the fluid from the flow feeder and percutaneously accessing a body, and a flow-limiting valve for cooperating with the driving mechanism and controlling an opening and closing of the first passage and the opening and closing of the second passage. The flow-limiting valve has a first state and a second state, the driving mechanism maintains the flow feeder at a first volume when the flow-limiting valve is in the first state, and the flow feeder receives a predetermined volume of fluid from the reservoir via the first passage; the driving mechanism maintains the flow feeder at a second volume when the flow-limiting valve is in the second state, and the flow feeder provides the predetermined volume of fluid via the second passage, the second volume is smaller than the first volume and the predetermined volume is determined by a volume difference between the first volume and the second volume.

In this case, it may be convenient for the patient to carry a large dose of the fluid for injection in case of need for treatment through storing the fluid in the reservoir of the medical device; by means of the cooperation between the flow feeder, the driving mechanism, and the flow-limiting valve, a predetermined volume of fluid is obtained from the reservoir via the first passage, i.e., the difference value between the first volume and the second volume of the flow feeder, the predetermined volume of fluid is infused into the body via the second passage, whereby it is able to obtain a predetermined volume of fluid for transfusion via the medical device when the patient needs the injection of fluid so as to achieve the effect of digital quantity control, resulting in improving the accuracy of the transfusion.

According to an embodiment of the disclosure, optionally, when the flow-limiting valve is in the first state, the flow-limiting valve opens the first passage and closes the second passage; when the flow-limiting valve is in the second state, the flow-limiting valve closes the first passage and opens the second passage. In this case, when the first passage is opened and the second passage is closed, the flow feeder communicating with the first passage may change its volume to a first volume driven by the driving mechanism so as to form a negative pressure and may obtain fluid from the reservoir communicating with the first passage via the first passage; when the first passage is closed and the second passage is open, the flow feeder communicating with the second passage may be restored to its original volume, i.e., the second volume, and may infuse the fluid into the body via the second passage.

According to an embodiment of the disclosure, optionally, the volume of the reservoir is variable, and when the reservoir contains the fluid, the pressure intensity of the fluid of the reservoir is maintained within a predetermined range, with the pressure intensity of the fluid of the reservoir being greater than the pressure intensity of the fluid of the flow feeder. In this case, the fluid is stored in the reservoir and maintained within a predetermined pressure intensity range, enabling the fluid to flow through the first passage into the flow feeder more easily, and serving to prevent fluid backflow or the occurrence of the blood entry resulting in a contaminated fluid.

According to an embodiment of the disclosure, optionally, the reservoir has a sealable supply port. In this case, the reservoir may be periodically replenished with fluid through the supply port; the sealable supply port may maintain the pressure intensity inside the reservoir or reduce the situation of air entering or contamination incurred by the external environment when the reservoir is replenished with the fluid.

According to an embodiment of the disclosure, optionally, the flow-limiting valve includes a first valve controlling the opening and closing of the first passage and a second valve controlling the opening and closing of the second passage. The second valve opens the second passage if the first valve closes the first passage, and the second valve closes the second passage if the first valve opens the first passage. In this case, when the first valve opens the first passage and the second valve closes the second passage, the flow feeder in communication with the first passage may be driven by the driving mechanism to change its volume to the first volume so as to form a negative pressure and may obtain fluids from the reservoir in communication with the first passage via the first passage; when the first valve closes the first passage and the second valve opens the second passage, the flow feeder in communication with the second passage may be restored to the original volume, i.e., a second volume, and may infuse the fluid into the body via the second passage; therefore, with continuously changing the first volume and the second volume, the flow feeder may continuously obtain a predetermined volume of the fluid and inject the same into the body via the second passage.

According to an embodiment of the disclosure, optionally, the driving mechanism includes a first driving mechanism and a second driving mechanism. The first driving mechanism controls the flow-limiting valve to open and close the first passage or the second passage, and the second driving mechanism controls the volume change of the flow feeder to switch the volume of the flow feeder between the first volume and the second volume. In this case, through the cooperation of the first driving mechanism and the second driving mechanism, the first driving mechanism drives the flow-limiting valve to be in a first state, i.e., when the first valve opens the first passage and the second valve closes the second passage, the second driving mechanism may control the volume of the flow feeder to be maintained at the first volume, whereby the flow feeder may form a negative pressure and obtain fluid from the reservoir via the first passage; when the fluid fills the first volume and the first driving mechanism drives the flow-limiting valve to be in the second state, i.e., when the first valve closes the first passage and the second valve opens the second passage, the second driving mechanism is capable of controlling the flow feeder volume to be maintained at the second volume, whereby the flow feeder is capable of infusing a predetermined volume of fluid into the body.

According to an embodiment of the disclosure, optionally, when the first driving mechanism keeps the flow-limiting valve in the first state, the flow-limiting valve opens the first passage and closes the second passage, and the second driving mechanism controls the flow feeder to maintain the first volume and receive the predetermined volume of fluid from the reservoir via the first passage; when the first driving mechanism keeps the flow-limiting valve in the second state, the flow-limiting valve closes the first passage and opens the second passage, and the second driving mechanism controls the flow feeder to maintain the second volume and provides the predetermined volume of fluid via the second passage. In this case, the flow-limiting valve is controlled to switch between the first state and the second state by the first driving mechanism, and the flow feeder is controlled to switch between the first volume and the second volume by cooperating with the second driving mechanism, whereby a predetermined volume of fluid may be continuously obtained and infused into the body, achieving quantitative or digital control, improving the control accuracy of the transfusion.

According to an embodiment of the disclosure, optionally, the flow-limiting valve is of a columnar body and has a first through hole and a second through hole; the first driving mechanism includes a first driver, and a first connector driven by the first driver and connected to the flow-limiting valve; if the first driver drives the first connector so as to rotate the flow-limiting valve to be in the first state, the first through hole is in communication with the first passage, and the second through hole is not in communication with the second passage; if the first driver drives the first connector so as to rotate the flow-limiting valve to be in the second state, the first through hole is not in communication with the first passage, and the second through hole is in communication with the second passage. In this case, via the flow-limiting valve which is of the columnar body, when driven by the first driving mechanism, the first through hole may be in communication with the first passage and the second through hole may be not in communication with the second passage, or the first through hole may be not in communication with the first passage and the second through hole may be in communication with the second passage; therefore, the flow-limiting valve which is of the columnar body may control the flow or non-flow of the fluid via the first passage or the second passage, while the inconvenience of designing multiple valves to control multiple passages may be reduced; in addition, the first driver may provide and transmit power to the flow-limiting valve through the first connector, thereby controlling the flow-limiting valve to switch between the first state and the second state.

According to an embodiment of the disclosure, optionally, the first through hole has a first axis and the second through hole has a second axis, the first axis being orthogonal to the second axis. In this case, by rotating the flow-limiting valve which is of the columnar body, for example by degrees clockwise or counterclockwise, the first through hole may be brought into communication with the first passage and the second through hole may be brought out of communication with the second passage, or the first through hole may be brought out of communication with the first passage and the second through hole may be brought into communication with the second passage,, and therefore, it is able to achieve to the effect of controlling the opening and closing of the two passages at the same time by one power.

According to an embodiment of the disclosure, optionally, one side of the reservoir is open and the opening is sealed by a first piston, and the other side of the reservoir is in communication with the first passage. In this case, it is able to push the fluid out of the first passage through the piston and maintain a predetermined pressure intensity when the fluid is contained in the reservoir so as to prevent a backflow of the fluid or the blood back flowing and contaminating the fluid in the reservoir.

According to an embodiment of the disclosure, optionally, the first piston is connected to a resilient component, the resilient component being maintained in a compressed state. In this case, the resilient component maintained in the compressed state may drive the first piston to displace so as to maintain the fluid contained in the reservoir at a predetermined pressure intensity, preventing the fluid backflow or blood from back flowing and contaminating the fluid in the reservoir.

According to an embodiment of the disclosure, optionally, the second driving mechanism includes a second driver, and a second connector driven by the second driver and connected to the flow feeder; if the second driver drives the second connector to change the volume of the flow feeder to maintain the first volume, the fluid flows from the reservoir to the flow feeder via the first passage, and if the second driver drives the second connector to change the volume of the flow feeder to maintain the second volume, the fluid flows from the flow feeder to the body via the second passage. In this case, the second driver provides power to be transmitted to the flow feeder via the second connector to change the volume of the flow feeder, thereby enabling a predetermined volume of fluid to be obtained from the reservoir and infused into the body in cooperation with the first driving mechanism and the flow-limiting valve.

According to an embodiment of the disclosure, optionally, one side of the flow feeder is open and the opening is sealed by a second piston, and the other side of the flow feeder is in communication with the second passage. In this case, the volume of the flow feeder may be changed by the displacement of the second piston and a negative pressure may be created at the first volume and a predetermined volume of the fluid may be obtained, the fluid is infused into the body through the second passage when a positive pressure is returned at the second volume.

According to an embodiment of the disclosure, optionally, the second piston is connected to the second connector and driven by the second driver. In this case, the second driver provides power to be transmitted to the second piston via the second connector; therefore, the volume of the flow feeder may be changed by the displacement of the second piston.

According to an embodiment of the disclosure, optionally, the first driver or the second driver is one of a shape memory alloy, a piezoelectric motor, and a servo motor, the first connector is one of a torsion spring and a gear, and the second connector is one of a spring and a connecting rod. In this case, the shape memory alloy is heated to a predetermined range by powering on so as to obtain the power generated by the deformation of the shape memory alloy so that it is able to drive the first connector and the flow-limiting valve; when the heating is stopped, it may recover the force so that the first connector and the flow-limiting valve may be driven reversely, which may have the advantages of power saving, accurate control and fast response; a piezoelectric motor or a servo motor drives the first connector and the flow-limiting valve, which may have the advantages of accurate control and fast response; in addition, the first connector constituted by a torsion spring or a gear may rotate and revolve the flow-limiting valve under power drive, thereby opening and closing the first passage or the second passage; in addition, the second connector constituted by a spring or a push rod may reciprocally displace the second piston under power drive, thereby changing the volume of the flow feeder.

The second aspect of the disclosure provides a medical system including: the medical device according to any of the first aspect of the disclosure.

According to the medical system to which the second aspect of the disclosure relates, optionally, the medical system further includes an external controller that controls the medical device and a sensing monitor communicatively connected to the external controller. The sensing monitor obtains data of a physiological parameters of a user and sends the data of the physiological parameters of the user to the external controller, the external controller controlling the medical device to deliver fluids based on the data of the physiological parameters of the user. In this case, the medical system may accurately provide transfusion therapy to the patient; in addition, the transfusion may be controlled by the external controller and the physiological parameters of the patient may be monitored by the sensing monitor, thereby enabling the medical system to provide transfusion therapy to the patient more accurately and automatically.

### BRIEF DESCRIPTION OF THE DRAWINGS

Illustrative embodiments of the disclosure are described in detail below with reference to the attached drawing figures.

FIG. 1 is a schematic diagram illustrating an application scenario of an embodiment of a medical system according to an example of the disclosure.

FIG. 2 is an overall schematic diagram showing a medical device in a medical system according to an example of FIG. 1 of the disclosure.

FIG. 3 is a schematic diagram showing the structure of a medical device according to an example of the disclosure.

FIG. 4 is a schematic diagram showing the structure of a reservoir of a medical device according to an example of the disclosure.

FIG. 5a is a schematic diagram illustrating the flow direction of the fluid in a medical device when a flow-limiting valve is in a first state and a flow feeder is at a first volume according to an example of the disclosure.

FIG. 5b is a schematic diagram illustrating the flow direction of the fluid in a medical device when a flow-limiting valve is in a second state and a flow feeder is at a second volume according to an example of the disclosure.

FIG. 6a is a cross-sectional diagram showing a flow-limiting valve (in a first state) of a medical device being connected to a first passage and a second passage according to an example of the disclosure.

FIG. 6b is a cross-sectional diagram showing a flow-limiting valve (in a second state) of a medical device being connected to a first passage and a second passage according to an example of the disclosure.

FIG. 7 is a schematic diagram illustrating the process of the rotation of a flow-limiting valve of a medical device from a first state to a second state according to an example of the disclosure.

FIG. 8 is a schematic diagram showing the structure of a flow feeder of a medical device according to an example of the disclosure.

FIG. 9 is a schematic diagram illustrating a flow feeder of a medical device at a first volume according to an example of the disclosure.

FIG. 10 is a schematic diagram illustrating a flow feeder of a medical device at a second volume according to an example of the disclosure.

FIG. 11 is a flowchart illustrating a control method of a medical device according to an example of the disclosure.

### DETAILED DESCRIPTION

The following describes some non-limiting exemplary embodiments of the invention with reference to the accompanying drawings. The described embodiments are merely a part rather than all of the embodiments of the invention. All other embodiments obtained by a person of ordinary skill in the art based on the embodiments of the disclosure shall fall within the scope of the disclosure.

It needs to be noted that the terms “first”, “second”, “third”, “fourth”, and the like in the description and the claims of the disclosure, as well as in the above-mentioned drawings, are used for distinguishing between different objects and not for describing a particular sequence. Furthermore, the terms “comprise” and “have”, as well as any variations thereof, are intended to cover a non-exclusive inclusion. For example, a process, method, system, product, or device that comprises a list of steps or units is not limited to the listed steps or units, but may optionally further comprise steps or units not listed, or may optionally further comprise other steps or units inherent to such process, method, product, or device. In the following description, the same components are given the same symbols such that the repeated description is omitted. In addition, the drawings are merely schematic, and the proportions of the dimensions of the components relative to each other, or the shapes of the components, etc. may differ from reality.

The disclosure relates to a medical device and a medical system for fluid conveyance. In the disclosure, a medical device for conveying a fluid may be referred to simply as a medical device or a device. A patient may refer to a user of a medical device to which the disclosure relates; the disclosure is not intended to be limiting with respect to the terms of “user”, “patients”, or “patient”, or the like, i.e., the meaning of the preceding words may sometimes be equivalent. Similarly, “fluid conveyance”, “transfusion”, “injected fluid”, and the like are not intended to be limiting and may be construed in the same or similar sense unless otherwise specifically limited.

In addition, in the disclosure, the fluid is not particularly limited and may be, for example, a medical solution injected through a medical device or a medical system to which the disclosure relates; in some examples, such medical solutions may be dopamine, dobutamine, adrenaline, noradrenaline bitartrate, sodium nitroprusside, stilamin, propofol, insulin, glucagon-like peptide-1, etc. In addition, the medical device to which the disclosure relates may also be used for regular, continuous, and precise drug delivery to a patient in conjunction with the actual situation of any disease.

One aspect of the disclosure relates to a medical system for automatic drug delivery; the medical system may include any of the medical devices (described later) according to another aspect of the disclosure.

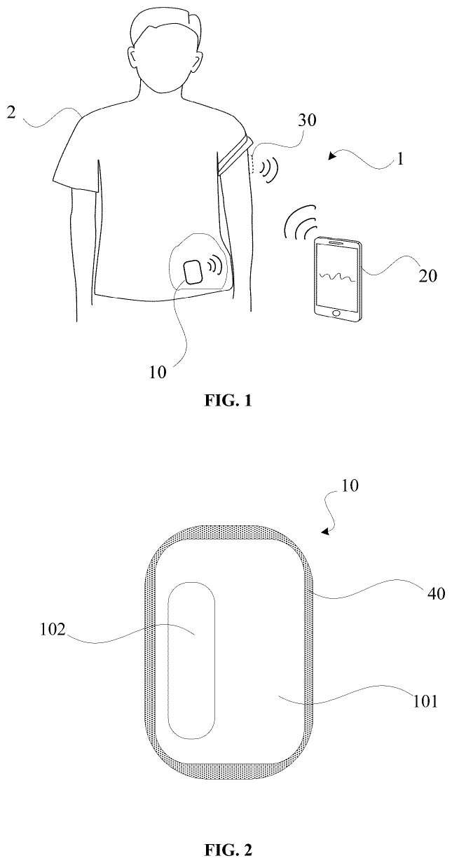

FIG. 1 is a schematic diagram illustrating an application scenario of an embodiment of a medical system 1 to which an example of the disclosure relates.

As shown in FIG. 1, in some examples, a medical system 1 for automatic drug delivery may include any of the medical devices 10 (described later) according to another aspect of the disclosure. In some examples, the medical system 1 may include devices such as a medical device 10, an external controller 20, and a sensing monitor 30. The medical system 1 for automatic drug delivery may hereinafter be simply referred to as the medical system 1 or the system 1. In some examples, the medical device may act on user 2 and may provide user 2 with transfusion therapy with quantitative control and high control accuracy.

In some examples, the external controller 20 may be a terminal apparatus such as a dedicated controller, a mobile phone, a personal computer, etc.; in some examples, the external controller 20 may also be a cloud apparatus or an Internet apparatus, for example, a server or a control terminal, etc. of an Internet hospital may be used as the external controller 20.

In some examples, the sensing monitor 30 may be a device used for monitoring a physiological parameters of a user, such as blood oxygen saturability, pulse rate, body temperature, height/weight, body composition, blood lipids, blood glucose, blood pressure, etc. This may not be limited herein.

In some examples, the sensing monitor 30 may be in wireless communication with the external controller 20. In some examples, wireless communication may include, but is not limited to, at least one of Bluetooth, Wi-Fi, 3G/4G/5G, NFC, UWB, and Zig-Bee.

In some examples, the external controller 20 may be in wireless communication with the medical device 10. In some examples, wireless communication may include, but is not limited to, at least one of Bluetooth, Wi-Fi, 3G/4G/5G, NFC, UWB, and Zig-Bee. In other examples, the external controller 20 may not be provided and the medical device 10 may have an internal controller or a control chip (not shown) that may be communicatively connected to the sensing monitor 30. In this case, the medical device 10 may directly respond to the monitoring data of the sensing monitor 30 and automatically provide the transfusion therapy to a patient in time, thereby improving the convenience and rapidity of the therapy.

In some examples, the sensing monitor 30 may obtain data of physiological parameters of the user and send the data to the external controller 20; the external controller 20 may control the medical device 10 to convey the fluid based on the data of the physiological parameters of the user.

In some examples, the sensing monitor 30 may be a blood glucose monitor by taking the treatment of diabetes as an example. The data of the physiological parameters of user 2 may be blood glucose data. The workflow of the medical system 1 may include: obtaining and sending real-time blood glucose data from the blood glucose monitor (i.e., the above-mentioned sensing monitor 30) of the user 2 to the external controller 20; and the external controller 20 sending a control signal to the medical device 10 20 based on the blood glucose data; the medical device 10 delivering fluid into the body of the user 2 based on the control signal.

Therefore, according to the disclosure, it is able to provide a medical system for conveying a medical solution with high precision.

Another aspect of the disclosure relates to a medical device for conveying a medical solution with high precision. In some examples, the medical device may be used to form any of the above-mentioned medical systems of the disclosure with other devices or instruments, that is, the medical device to which the disclosure relates may be one of the constituent elements of any of the above-mentioned medical systems.

FIG. 2 is an overall schematic diagram showing a medical device 10 in a medical system 1 to which an example of FIG. 1 of the disclosure relates. FIG. 3 is a schematic diagram showing the structure of a medical device 10 to which an example of the disclosure relates.

As shown in FIG. 3, a medical device 10 according to the disclosure may include: a reservoir 11, a first passage 12, a flow feeder 13, a driving mechanism 15, a second passage 14, and a flow-limiting valve 16.

In some examples, the reservoir 11 may be used to contain fluid, the first passage 12 may be in communication with the reservoir 11, the flow feeder 13 may be used to receive fluid from the reservoir 11 via the first passage 12 and provide the fluid, the driving mechanism 15 may be used to control the volume change of the flow feeder 13, the second passage 14 may be in communication with the flow feeder 13 and receive fluid from the flow feeder 13 and percutaneously access a body, and the flow-limiting valve 16 may be used in conjunction with the driving mechanism 15 and may be used to control the opening and closing of first passage 12 and the opening and closing of the second passage 14.

In some examples, the flow-limiting valve 16 may have a first state and a second state; when the flow-limiting valve 16 is in the first state, the driving mechanism 15 may maintain the flow feeder 13 at a first volume, and the flow feeder 13 may receive a predetermined volume of fluid from the reservoir 11 via the first passage 12; when the flow-limiting valve 16 is in the second state, the driving mechanism 15 may maintain the flow feeder 13 at a second volume, the flow feeder 13 may provide a predetermined volume of fluid via the second passage 14, the second volume may be smaller than the first volume, and the predetermined volume may be determined by a volume difference between the first volume and the second volume.

In this case, through storing the fluid in the reservoir 11 of the medical device 10, it is convenient for the patient to carry a predetermined dose of the fluid for injection in case of need so that the patient may receive treatment in time; by means of the cooperation between the flow feeder 13, the driving mechanism 15, and the flow-limiting valve 16, a predetermined volume of fluid is obtained from the reservoir 11 via the first passage 12, i.e., the difference value between the first volume and the second volume of the flow feeder 13, and the predetermined volume of fluid is infused into the body via the second passage 14, whereby it is able to obtain a predetermined volume (i.e., a unit quantity or a base quantity) of fluid for transfusion via the medical device 10 when the patient needs the injection of fluid so as to achieve the effect of digital quantity control, thereby improving the accuracy of the transfusion. In some examples, the predetermined volume of fluid may also be referred to as a unit quantity, a base quantity, or a digital quantity of fluid, and the predetermined volume of fluid may be determined by varying the first volume or the second volume of the flow feeder 13 according to different fluid types and treatment effects, for example, according to the scenario that diabetic patients need specific therapeutic effects, the quantity of insulin of a predetermined volume obtained each time may be set as 0.1 mg (or ml), 0.5 mg (or ml), 1 mg (or ml), etc.

As shown in FIG. 3, in some examples, the medical device 10 may also include a control chip 17 used for responding to an external control signal from an external controller 20 (see FIG. 1), and the control chip 17 may send a control instruction to control the driving mechanism 15 based on the external control signal.

In some examples, control chip 17 may include a control module and a communication module. The control module (not shown) may be used to generate a control instruction based on the external control signal to control the driving mechanism 15, for example, start, pause, continue, or stop controlling. The communication module (not shown) may be used to do communication and data interaction with the external controller 20, for example, receiving an external control signal or sending the working state information, etc. of the current medical device 10.

As described above, the medical device 10 may infuse fluid into a patient based on a control signal. Specifically, the medical device 10 may generate a control instruction based on the control signal and control the driving mechanism 15 to drive the flow-limiting valve 16 and flow feeder 13 to obtain a predetermined volume of fluid and infuse the fluid into the patient. Therefore, it is able to achieve the effect of precise control of the digital quantity and improve the accuracy of the transfusion.

In some examples, the control chip 17 of the medical device 10 may be directly responsive to the data of a physiological parameter of a human body monitored by the sensing monitor 30 (see FIG. 1) and control the transfusion of fluid into the patient based on the data. In this case, the response time may be reduced compared to the aforementioned process of controlling the transfusion in response to the external control signal of the external controller 20 so that the patient may be more quickly assisted in getting treatment.

As shown in FIG. 3, in some examples, the medical device 10 may also include a battery module 18. The battery module 18 may be used to provide power to the driving mechanism 15 and the control chip 17. In some examples, the battery module 18 may also be used to provide power to other modules in the medical device 10 that require electric power. In some examples, the battery module 18 may be an energy module having an independent power source such as a lithium battery, a button battery, or the like. In other examples, the battery module 18 may be an energy storage module that is charged by an interface such as a USB or Type-C.

As shown in FIG. 3, in other examples, a medical device 10 for delivering fluids may include: a reservoir 11, a first passage 12, a flow feeder 13, a driving mechanism 15, a second passage 14, a flow-limiting valve 16, a needle assisting device 19, and a filter 104.

In some examples, a needle assisting device 19 may be used to insert the extension of the second passage 14 subcutaneously. Thereby, the medical device 10 may infuse fluids into the body through the second passageway 14.

In some examples, the subcutaneously inserted portion of the needle assisting device 19 may be a needle or a trocar, the needle assisting device 19 may advance the extension of the second passage 14 subcutaneously in a single pass and withdraw the needle or trocar leaving the extension of the second passage 14 subcutaneously. Thus, the medical device 10 may infuse fluids into the body through the second passageway 14 and may reduce the patient's pain from multiple needle sticks.

In some examples, the medical device 10 may not include the needle assisting device 19. In this case, the medical device 10 may be connected to the transfusion tube provided in the patient's body through the second passage 14 and the transfusion may be accomplished, thereby enabling the patient who does not easily receive the drug carried in the application mode to perform the transfusion or facilitating the use in different scenarios.

In some examples, a filter 104 may be provided between the reservoir 11 and the first passage 12. The filter 104 may be configured to filter the fluid prior to the fluid provision of the reservoir 11 to the first passage 12. In this case, it is able to reduce a clogging situation caused by possible crystallization of the fluid (e.g., a drug such as insulin), thereby allowing the fluid to smoothly flow into the flow feeder 13 and improving the accuracy of fluid conveyance into the patient. In other examples, the medical device 10 may not be provided with a filter 104, for example, t the filter 104 may not be provided as the fluid is a medical fluid that does not easily crystallize.

In other examples, the medical device 10 for conveying fluids may also include an alarm device (not shown). The alarm device may be configured to detect the condition of the fluid in the reservoir 11. When the fluid storage amount is less, an alarm information may be sent, for example, a vibration or a sound, to prompt the patient to supply.

Refer to FIG. 2 or 3, in some examples, the medical device 10 may include a housing 101. The housing 101 may serve as a housing for the medical device 10 to protect the internal components of the medical device 10. In some examples, the housing 101 may be designed as shape of a flat prolonged block with an arc that may be adapted to the skin surface. In some examples, one surface of the housing 101 may be assembled with an application film 40 for attaching to the skin, i.e., the medical device 10 may be of the application type. In this case, the medical device 10 of the application type may be conveniently carried on by the patient and, in addition, may be used to treat patients by transfusion in time in the event of an emergency.

In some examples, the housing 101 may be provided with a transparent window 102 that visually displays the fluid volume of the reservoir 11 (see FIG. 2), thereby enabling the patient to view and replenish the fluid in a timely manner.

In other examples, the housing 101 of the medical device 10 may be provided as shape of a non-flat prolonged block (e.g., a sphere or a cube), that is, the housing 101 may be provided as a general block, and the medical device 10 may not be assembled with the application film 40. In some examples, a housing provided as a general block may be provided with a hanging structure or mechanism or a structure or mechanism easy for hand-holding. In this case, the medical device may be conveniently used for the treatment in the ward, or transfusion treatment may be conveniently performed by some users who do not easily receive the way of carrying a drug by application.

As described above, the outline configuration of the housing 101 of the medical device 10 may not be limited, in other words, the housing 101 of the medical device 10 may be an applied or an externally non-applied device. In other examples, the medical device 10 may be provided to be an implanted subcutaneous or intracorporeal device, and the housing 101 may be formed into a particular shape by using a material that is biocompatible, for example, a device that accommodates different body parts, such as an implantable antinociceptive pump, a hepatic vascular completely implantable drug pump, etc.

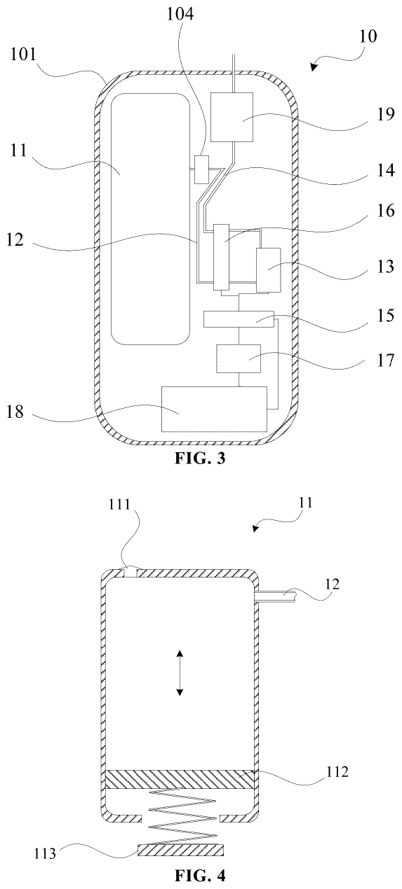

FIG. 4 is a schematic diagram showing the structure of a reservoir 11 of a medical device 10 to which an example of the disclosure relates.

As shown in FIG. 4, in some examples, the volume of the reservoir 11 may be variable, and the pressure intensity of the fluid of the reservoir 11 may be maintained within a predetermined range when the reservoir 11 accommodates the fluid. The pressure intensity of the fluid of the reservoir 11 may be greater than the pressure intensity of the fluid of the flow feeder 13. In this case, the fluid is stored in the reservoir 11 and maintained within a predetermined pressure intensity range, enabling the fluid to flow more easily through the first passage 12 into the flow feeder 13, and serving to reduce fluid backflow or the occurrence of the blood entering the medical device 10 resulting in a contaminated fluid.

In some examples, the reservoir 11 may have a sealable supply port 111. In this case, the reservoir 11 may be periodically replenished with fluid through the supply port 111; in addition, the sealable supply port 111 may maintain the pressure intensity inside the reservoir 11 or reduce the situation of the air entrance or contamination caused by the external environment as the reservoir 11 is replenished with the fluid. In some examples, the location of the supply port 111 at the reservoir 11 may not be limited. In some examples, supply port 111 may cooperate with an external conduit to supply fluid to reservoir 11.

In some examples, one side of the reservoir 11 may be open and sealed by a first piston 112 and the other side of the reservoir 11 may be in communication with the first passage 12. In this case, it is able to push the fluid out of the first passage 12 through the piston and maintain a predetermined pressure intensity when the fluid is accommodated in the reservoir 11 so that it is able to reduce the backflow of the fluid or a situation of the blood flowing into the medical device 10 result in contaminating the fluid in the reservoir 11.

In some examples, the first piston 112 may be connected to a resilient component 113, the resilient component 113 may be maintained in a compressed state. In this case, the resilient component 113 maintained in the compressed state may drive the first piston 112 to displace so as to maintain the fluid contained in the reservoir 11 at a predetermined pressure intensity, thereby reducing the fluid backflow or a situation of blood backflowing to contaminate the fluid in the reservoir 11, etc.

In other examples, the reservoir 11 may also be a retractable container made of a resilient material capable of maintaining the pressure intensity within a predetermined range as accommodating the fluid. For example, silica gel, rubber, and the like. In this case, the above-mentioned effect may be achieved without providing the first piston 112 and the resilient component 113.

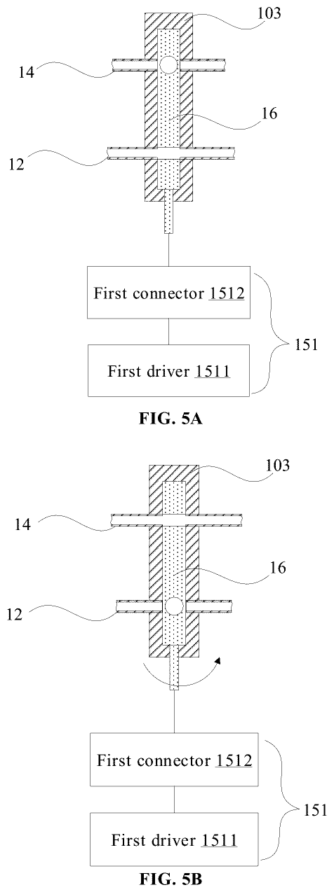

As mentioned above, the medical device 1 may include a flow-limiting valve 16. FIG. 5a is a cross-sectional diagram showing a flow-limiting valve 16 (in a first state) of a medical device 10 being connected to a first passage 12 and a second passage 14 according to an example of the disclosure. FIG. 5b is a cross-sectional diagram showing a flow-limiting valve 16 (in a second state) of a medical device 10 being connected to a first passage 12 and a second passage 14 according to an example of the disclosure.

Refer to FIGS. 5a and 5b, in some examples, as the flow-limiting valve 16 is in the first state, the flow-limiting valve 16 may open the first passage 12 and close the second passage 14; as the flow-limiting valve 16 is in the second state, the flow-limiting valve 16 may close the first passage 12 and open the second passage 14. In this case, as the first passage 12 is opened and the second passage 14 is closed, the flow feeder 13 communicating with the first passage 12 may be driven to change the volume to a first volume by the driving mechanism 15, and a negative pressure may be formed in the flow feeder 13 to obtain fluid from the reservoir 11 communicating with the first passage 12 via the first passage 12; as the first passage 12 is closed and the second passage 14 is opened, the volume of the flow feeder 13 communicating with the second passage 14 may be restored to its original volume, i.e., the second volume, and the fluid may be infused into the body via the second passage 14.

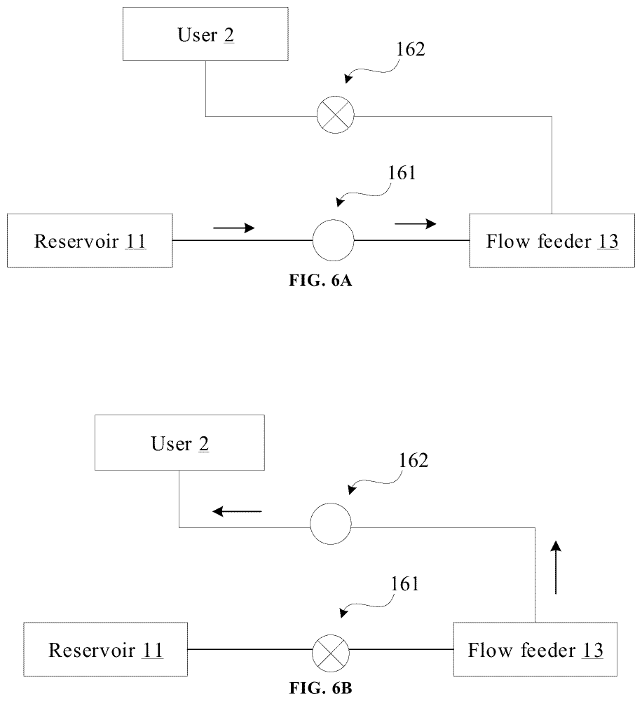

FIG. 6a is a schematic diagram illustrating the flow direction of the fluid in a medical device 10 as a flow-limiting valve 16 is in a first state and a flow feeder 13 is at a first volume according to an example of the disclosure; FIG. 6b is a schematic diagram illustrating the flow direction of the fluid in a medical device 10 as a flow-limiting valve 16 is in a second state and a flow feeder 13 is at a second volume according to an example of the disclosure.

As shown in FIGS. 6a and 6b, in some examples, the flow-limiting valve 16 may include a first valve 161 controlling the opening and closing of the first passage 12 and a second valve 162 controlling the opening and closing of the second passage 14; as the first valve 161 closes the first passage 12, the second valve 162 opens the second passage 14, as the first valve 161 opens the first passage 12, the second valve 162 closes the second passage 14. In this case, as the first valve 161 opens the first passage 12 and the second valve 162 closes the second passage 14, the volume of the flow feeder 13 in communication with the first passage 12 may be driven by the driving mechanism 15 to change to the first volume so that negative pressure may be formed in to obtain fluids from the reservoir 11 in communication with the first passage 12 via the first passage 12; as the first valve 161 closes the first passage 12 and the second valve 162 opens the second passage 14, the volume of the flow feeder 13 in communication with the second passage 14 may be restored to the original volume, i.e., a second volume, and the fluid may be infused into the body via the second passage 14; thereby, through continuously interchanging of the first volume and the second volume, the flow feeder 13 may continuously obtain and infuse a predetermined volume (i.e. unit quantity or base quantity) of the fluid into the body.

In some examples, the first valve 161 and the second valve 162 may be provided as one of a stop-valve-shaped valve, a cock-shaped (or spherical) valve, a gate-shaped valve, a swing-shaped valve, a butterfly-shaped valve, and a slide valve-shaped valve.

In some examples, the first valve 161 and the second valve 162 may be controlled simultaneously, that is, driven by the identical power source (e.g., the first driving mechanism 151 below). In this case, the flow feeder 13 obtains the predetermined volume of the fluid by simultaneously controlling the first valve 161 to open and the second valve 162 to close, or controlling the first valve 161 to close and the second valve 162 to open; therefore, the accuracy in obtaining the predetermined volume of the fluid by the flow feeder 13 may be improved.

In other examples, the first valve 161 and the second valve 162 may be independently controlled, that is, driven by different power sources. In this case, the first valve 161 and the second valve 162, which are independently controlled, may also have the aforementioned effect of controlling the first valve 161 to open and the second valve 162 to close, or controlling the first valve 161 to close and the second valve 162 to open so that the flow feeder 13 obtains a predetermined volume of fluid, and the delay in fluid flow is calculated into the control by combining a mathematical calculation, therefore, the accuracy of obtaining a predetermined volume of fluid by the flow feeder 13 may also be improved.

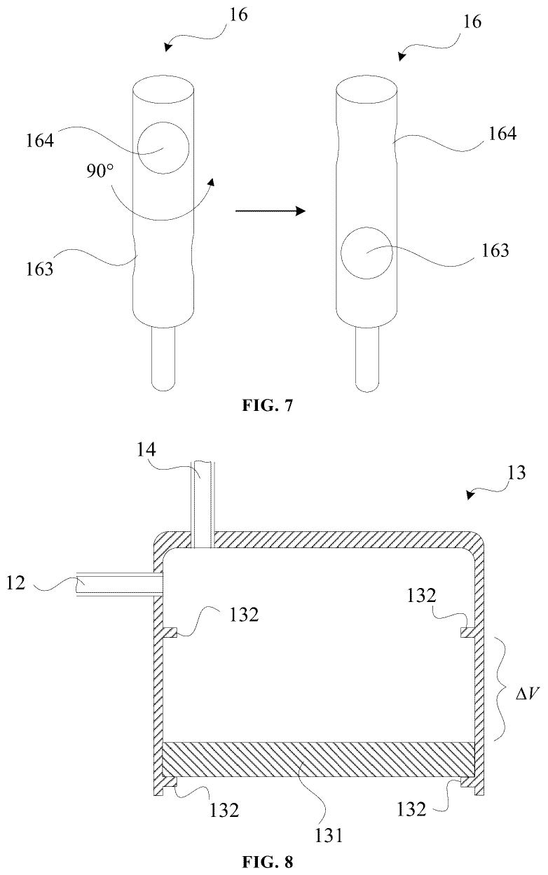

FIG. 7 is a schematic diagram illustrating the process of the rotation of a flow-limiting valve 16 of a medical device 10 from a first state to a second state according to an example of the disclosure.

As mentioned above, in some examples, the medical device 10 may include a driving mechanism 15 (described later), the driving mechanism 15 may include a first driving mechanism 151 (see FIGS. 5a and 5b).

In some examples, the flow-limiting valve 16 may be rotatably wrapped within the sealing material body 103 and may have an extension portion (not shown), the extension portion may be connected to and driven by the first driving mechanism 151 (see FIGS. 5a and 5b). A sealing material body 103 may form a liquid flow cavity channel with the first passage 12 and the second passage 14 (see FIGS. 5a and 5b).

In an exemplary embodiment of the disclosure, the materials of the sealing material body 103, the first passage 12, and the second passage 14 may optionally be made of the same material, e.g., silica gel, or may be installed differently and designed to be made of different materials, e.g., the portion of the second passage 14 implanted into the patient may be made of a material with goods biocompatibility, e.g., polypropylene, siloxane, polyurethane, acrylic derivatives, polyhydroxy acids, etc.

As shown in FIGS. 5a, 5b, and 7, in some examples, the flow-limiting valve 16 may be of a columnar body. In some examples, the flow-limiting valve 16 may have a first through hole 163 and a second through hole 164 (see FIG. 7). In some examples, the flow-limiting valve 16 may be columnar and have a first through hole 163 and a second through hole 164.

As shown in FIGS. 5a and 5b, in some examples, the first driving mechanism 151 may include a first driver 1511, and a first connector 1512 driven by the first driver 1511 and connected to the flow-limiting valve 16. As the first driver 1511 drives the first connector 1512 to rotate the flow-limiting valve 16 to be in the first state, the first through hole 163 communicates with the first passage 12 and the second through hole 164 does not communicate with the second passage 14, as the first driver 1511 drives the first connector 1512 to rotate the flow-limiting valve 16 to be in the second state, the first through hole 163 does not communicate with the first passage 12 and the second through hole 164 communicates with the second passage 14.

In this case, as the flow-limiting valve 16 of the columnar body is driven by the first driving mechanism 151, the first through hole 163 may communicate with the first passage 12 and the second through hole 164 may not be in communication with the second passage 14, or the first through hole 163 may not be in communication with the first passage 12 and the second through hole 164 may communicate with the second passage 14; thereby, the flow-limiting valve 16 of the columnar body may control the fluid to flow or not flow via the first passage 12 or the second passage 14, while the inconvenience of designing multiple valves to control multiple passages may be reduced. In addition, the first driver 1511 may provide and transmit power to the flow-limiting valve 16 through the first connector 1512, so as to control the flow-limiting valve 16 to switch between the first state and the second state.

As shown in FIG. 7, in some examples, the first through hole 163 may have a first axis, the second through hole 164 may have a second axis, and the first axis and the second axis may be orthogonal. In this case, rotating the flow-limiting valve 16 of a columnar body shape is rotated, for example, 90 degrees clockwise or counterclockwise, the first through hole 163 may be in communication with the first passage 12 and the second through hole 164 may not be in communication with the second passage 14, or the first through hole 163 may be in communication with the first passage 12 and the second through hole 164 may not be in communication with the second passage 14. Thereby, the effect of controlling the opening and closing of the two passages at the same time by one power may be achieved.

In some examples, the first axis and the second axis may intersect spatially but not be parallel. In this case, the first through hole 163 having the first axis and the second through hole 164 having the second axis also enable the first through hole 163 to communicate with the first passage 12 and the second through hole 164 to not communicate with the second passage 14 or the first through hole 163 to not communicate with the first passage 12 and the second through hole 164 to communicate with the second passage 14 as the flow-limiting valve 16 is rotated to a predetermined angle. That is, two states that the first through hole 163 and the first passage 12 are in communication and the second through hole 164 and the second passage 14 are not n communication, or the first through hole 163 and the first passage 12 are not n communication and the second through hole 164 and the second passage 14 are n communication are satisfied.

In some examples, the rotation of the flow-limiting valve 16 may be unidirectional or reciprocating. Preferably, the embodiment the disclosure employs the reciprocating rotation.

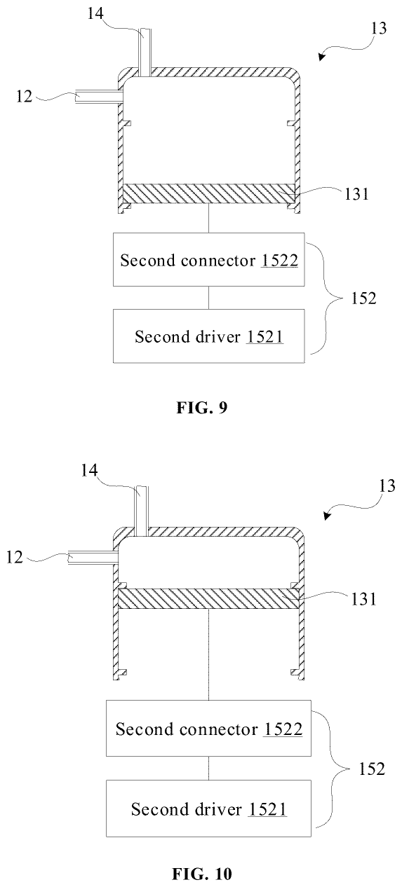

FIG. 8 is a schematic structural diagram showing a flow feeder 13 of a medical device 10 according to an example of the disclosure; FIG. 9 is a schematic diagram illustrating a flow feeder 13 of a medical device 10 at a first volume according to an example of the disclosure; FIG. 10 is a schematic diagram illustrating a flow feeder 13 of a medical device 10 at a second volume according to an example of the disclosure.

As described above, in some examples, the flow-limiting valve 16 may have a first state and a second state, as the flow-limiting valve 16 is in the first state, the driving mechanism 15 may maintain the flow feeder 13 at the first volume (see FIG. 9), and the flow feeder 13 may receive a predetermined volume of the fluid from the reservoir 11 via the first passage 12; as the flow-limiting valve 16 is in the second state, the driving mechanism 15 may maintain the flow feeder 13 at the second volume (see FIG. 10), the flow feeder 13 may provide a predetermined volume of fluid AV via the second passage 14, the second volume may be smaller than the first volume, and the predetermined volume AV may be equal to the first volume minus the second volume (see FIG. 8).

As shown in FIGS. 8, 9, and 10, in some examples, one side of the flow feeder 13 may be open and sealed by the second piston 131, and the other side of the flow feeder 13 may communicate with the second passage 14. In this case, the volume of the flow feeder 13 may be changed by the displacement of the second piston 131 and negative pressure may be formed at the first volume and a predetermined volume of the fluid may be obtained, and positive pressure may be recovered at the second volume and the fluid may be infused into the body through the second passage 14.

In some examples, the driving mechanism 15 may include a second driving mechanism 152, and the second piston 131 may be connected to the second connector 1522 and driven by the second driver 1521. In this case, the second driver 1521 provides and transmits power to the second piston 131 via the second connector 1522; thereby, the volume of the flow feeder 13 may be changed by the displacement of the second piston 131.

As shown in FIG. 8, in some examples, in order to ensure the accuracy of the volume change of the flow feeder 13, multiple limiting protrusions 132 may also be provided in the flow feeder 13. In this case, as the second piston 131 reciprocates in the flow feeder 13 to change the volume of the flow feeder 13, the limiting protrusions 132 may limit the second piston 131 to improve the accuracy of the volume change of the flow feeder 13.

In other examples, the flow feeder 13 may also be a retractable container formed of a resilient material, capable of expanding to a predetermined degree and creating a negative pressure when the filling of the fluid is required, and capable of retracting to the original shape to expel the fluid when the fluid is required to be expelled. In this case, the provision of the second piston 131 and the second connector 1522 may be reduced.

As the previously described driving mechanism 15, in some examples, the driving mechanism 15 may include the above-described first driving mechanism 151 and the above-described second driving mechanism 152. In some examples, the first driving mechanism 151 may control the flow-limiting valve 16 to open and close the first passage 12 or the second passage 14 (see FIGS. 5a and 5b, or FIGS. 6a and 6b), and the second driving mechanism 152 may control the volume change of the flow feeder 13 to switch the volume of the flow feeder 13 between the first volume and the second volume (see FIGS. 9 and 10).

In this case, through the cooperation of the first driving mechanism 151 and the second driving mechanism 152, the first driving mechanism 151 drives the flow-limiting valve 16 to be in the first state, that is, as the first valve 161 opens the first passage 12 and the second valve 162 closes the second passage 14, the second driving mechanism 152 may control the volume of the flow feeder 13 to be maintained at the first volume; thereby, a negative pressure may be formed in the flow feeder 13 and fluids may be obtained from the reservoir 11 via the first passage 12; as the fluid fills the first volume and the first driving mechanism 151 drives the flow-limiting valve 16 to be in the second state, that is, as the first valve 161 closes the first passage 12 and the second valve 162 opens the second passage 14, the second driving mechanism 152 may control the volume of the flow feeder 13 to be maintained at the second volume, thereby, the flow feeder 13 may infuse a predetermined volume of the fluid into the body.

In some examples, the first driving mechanism 151 and the second driving mechanism 152 may be controlled simultaneously, that is, as the first driving mechanism 151 controls the flow-limiting valve 16 to open and close the first passage 12 or the second passage 14, the second driving mechanism 152 simultaneously controls the volume change of the flow feeder 13 to switch the volume of the flow feeder 13 between the first volume and the second volume. In this case, the simultaneous control may reduce the time of the transfusion of the fluid into the body and reduce the number of sequential inputs of a control instruction, thereby enabling the simplification of a control program of a control chip 17 and enabling the speeding up of the treatment of a patient.

In some examples, the first driving mechanism 151 and the second driving mechanism 152 may be controlled in time delay manner, that is, as the first driving mechanism 151 controls the flow-limiting valve 16 to open and close the first passage 12 or the second passage 14, the second driving mechanism 152, following the control of the flow-limiting valve 16 by the first driving mechanism 151, controls the volume change of the flow feeder 13 to switch the volume of the flow feeder 13 between the first volume and the second volume. In this case, the time delayed control may improve the accuracy of the control and reduce the situations of poor treatment effect caused by wrong control sequences.

As shown in FIG. 5a or 6a, in some examples, when the first driving mechanism 151 enables the flow-limiting valve 16 to be at the first state, the flow-limiting valve 16 may open the first passage 12 and close the second passage 14, and the second driving mechanism 152 may control the flow feeder 13 to maintain the first volume and receive a predetermined volume of fluid from the reservoir 11 via the first passage 12. As shown in FIG. 5b or 6b, in some examples, when the first driving mechanism 151 enables the flow-limiting valve 16 to be at the second state, the flow-limiting valve 16 may close the first passage 12 and open the second passage 14, and the second driving mechanism 152 may control the flow feeder 13 to maintain the second volume and provide a predetermined volume of fluid via the second passage 14. In this case, the flow-limiting valve 16 is controlled to switch between the first state and the second state by the first driving mechanism 151, and the flow feeder 13 is controlled to switch between the first volume and the second volume by cooperating with the second driving mechanism 152, thereby, a predetermined volume of fluid may be continuously obtained and infused into the body, achieving quantitative or digital quantity control and improving the control accuracy of the transfusion.

As shown in FIG. 5a or 5b, in some examples, the second driving mechanism 152 may include a second driver 1521, and a second connector 1522 driven by the second driver 1521 and connected to the flow feeder 13.

In some examples, the fluid flows from the reservoir 11 to the flow feeder 13 via the first passage 12 if the second driver 1521 drives the second connector 1522 to change the volume of the flow feeder 13 to maintain the first volume, and fluid flows from the flow feeder 13 to the body via the second passage 14 if the second driver 1521 drives the second connector 1522 to change the volume of the flow feeder 13 to maintain the second volume. In this case, the second driver 1521 provides and transmits power to the flow feeder 13 via the second connector 1522 to change the volume of the flow feeder 13, thereby predetermined volume of fluid may be obtained from the reservoir 11 and infused into the body in cooperation with the first driving mechanism 151 and the flow-limiting valve 16.

In some examples, the first driver 1511 or the second driver 1521 may be one of a shape memory alloy, a piezoelectric motor, and a servo motor. The first connector 1512 may be one of a torsion spring and a gear. The second connector 1522 may be one of a spring and a connecting rod. In this case, the shape memory alloy is heated to a predetermined range by powering on so as to obtain the power generated by the deformation of the shape memory alloy so that it is able to drive the first connector 1512 and the flow-limiting valve 16, when the heating is stopped, the first connector 1512 and the flow-limiting valve 16 may be driven reversely when the power is generated by the deformation recovery of the shape memory alloy, which may have the advantages of power saving, accurate control and fast response. The first connector 1512 and the flow-limiting valve 16 are driven by a piezoelectric motor or a servo motor, which may have the advantages of accurate control and fast response; in addition, the first connector 1512 constituted by a torsion spring or a gear may rotate and revolve the flow-limiting valve 16 under power drive, thereby opening and closing the first passage 12 or the second passage 14; in addition, the second connector 1522 constituted by a spring or a push rod may reciprocally displace the second piston 131 under power drive, thereby changing the volume of the flow feeder 13.

In order to better embody the functional effects of the medical device 10 to which the disclosure relates, the disclosure may also provide a method for controlling a medical device 10 for fluid conveyance. Hereinafter, it may be simply referred to as a control method or a method.

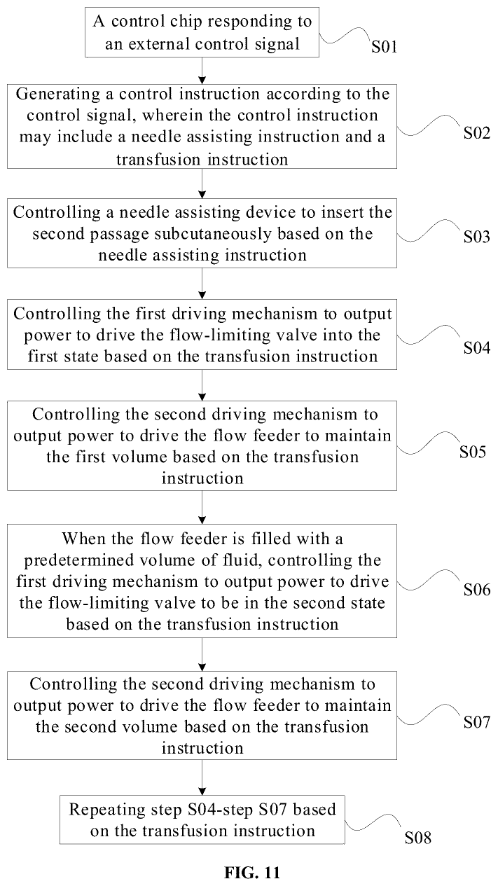

FIG. 11 is a flowchart illustrating a control method of a medical device 10 according to an example of the disclosure.

In some examples, as shown in FIG. 11, the control method may include: step S01: a control chip 17 responding to an external control signal.

Step S02: generating a control instruction according to the control signal. The control instruction may include a needle assisting instruction and a transfusion instruction, the needle assisting instruction is used for controlling the needle assisting device 19 to insert the second passage 14 subcutaneously prior to delivering the fluid and the transfusion instruction is used for controlling the driving mechanism 15.

Step S03: controlling the needle assisting device 19 to insert the second passage 14 subcutaneously based on the needle assisting instruction.

Step S04: based on the transfusion instruction, controlling the first driving mechanism 151 to output power to drive the flow-limiting valve 16 into a first state, that is, the first passage 12 is opened and the second passage 14 is closed.

Step S05: based on the transfusion instruction, controlling the second driving mechanism 152 to output power to drive the flow feeder 13 to maintain the first volume.

Step S06: when the flow feeder 13 is filled with a predetermined volume of fluid, based on the transfusion instruction, controlling the first driving mechanism 151 to output power to drive the flow-limiting valve 16 into the second state, that is, the first passage 12 is closed and the second passage 14 is opened.

Step S07: based on the transfusion instruction, controlling the second driving mechanism 152 to output power to drive the flow feeder 13 to maintain a second volume, thereby a predetermined volume of the fluid is infused into the body through the second passage 14. The second volume is smaller than the first volume, the predetermined volume is equal to the first volume minus the second volume.

Step S08: repeating step S04 to step S07 based on the transfusion instruction, thereby continuously providing a quantitative medical solution to the patient according to the required amount of medical solution and improving the therapeutic effect.

In some examples, steps S04 and S05 may be performed simultaneously, and steps S06 and S07 may be performed simultaneously.

In some examples, in step S04, the first driving mechanism 151 may output power reversely to drive the flow-limiting valve 16 in the second state, that is, the first passage 12 is closed and the second passage 14 is opened. In other words, the first driving mechanism 151 may drive the flow-limiting valve 16 to reciprocate so that the first passage 12 is switched between being opened and closed and the second passage 14 is switched between being closed and opened, that is, after step S04 is completed, step S06 may be completed by reciprocating motion.

In some examples, in step S05, the second driving mechanism 152 may output power reversely to maintain the flow feeder 13 at the second volume. In other words, the second driving mechanism 152 may drive the second piston 131 to reciprocate so that the volume of the flow feeder 13 is switched between the first volume and the second volume, that is, after step S05 is completed, step S07 may be completed by reciprocating motion.

According to another aspect of the disclosure, a medical device 10 for delivering fluids with high precision may be provided. The medical device controls the delivery of the fluid by a digital quantity (i.e., a predetermined volume of fluid) so that it may be featured with high precision of fluid conveyance in comparison with the prior art.

According to the above two aspects of the disclosure, it is able to provide a medical device and a medical system for conveying fluids with high precision.

Although the disclosure has been specifically described above with reference to the accompanying drawings and examples, it will be understood that the above description does not limit the disclosure in any form. Those skilled in the art may make modifications and variations to the disclosure as required without departing from the true spirit and scope of the disclosure, and these modifications and variations fall within the scope of the disclosure.

Various embodiments of the disclosure may have one or more of the following effects. In some embodiments, the effect of digital quantity control may be achieved by obtaining a predetermined volume of fluid for injection so that the fluid may be delivered with high precision. In other embodiments, the disclosure may be able to provide a medical device and a medical system for conveying fluids with high precision. The medical device controls the conveyance of the fluid by a digital quantity (i.e., a predetermined volume of fluid) so that it may be featured with high precision of fluid conveyance compared to the prior art

Many different arrangements of the various components depicted, as well as components not shown, are possible without departing from the spirit and scope of the disclosure. Embodiments of the disclosure have been described with the intent to be illustrative rather than restrictive. Alternative embodiments will become apparent to those skilled in the art that do not depart from its scope. A skilled artisan may develop alternative means of implementing the aforementioned improvements without departing from the scope of the disclosure.

It will be understood that certain features and subcombinations are of utility and may be employed without reference to other features and subcombinations and are contemplated within the scope of the claims. Unless indicated otherwise, not all steps listed in the various figures need be carried out in the specific order described.

The disclosure claimed is:

1.-17. (canceled)

18. A medical device for fluid conveyance, comprising:
a reservoir for containing a fluid;
a first passage in communication with the reservoir;
a flow feeder for receiving the fluid from the reservoir via the first passage and providing the fluid;
a driving mechanism for controlling a volume change of the flow feeder;
a second passage, in communication with the flow feeder, for receiving the fluid from the flow feeder and percutaneously accessing a body; and
a flow-limiting valve for cooperating with the driving mechanism, for controlling an opening and closing of the first passage, and for controlling the opening and closing of the second passage;
wherein:
the flow-limiting valve comprises a first state and a second state;
the driving mechanism maintains the flow feeder at a first volume when the flow-limiting valve is in the first state, and the flow feeder receives a predetermined volume of fluid from the reservoir via the first passage;
the driving mechanism maintains the flow feeder at a second volume when the flow-limiting valve is in the second state, and the flow feeder provides the predetermined volume of fluid via the second passage; and
the second volume is smaller than the first volume and the predetermined volume is determined by a volume difference between the first volume and the second volume.

19. The medical device according to claim 18, wherein:
when the flow-limiting valve is in the first state, the flow-limiting valve opens the first passage and closes the second passage; and
when the flow-limiting valve is in the second state, the flow-limiting valve closes the first passage and opens the second passage.

20. The medical device according to claim 18, wherein a volume of the reservoir is variable, and when the reservoir contains the fluid, a pressure intensity of the fluid of the reservoir is maintained within a predetermined range, with the pressure intensity of the fluid of the reservoir being greater than the pressure intensity of the fluid of the flow feeder.

21. The medical device according to claim 18, wherein the reservoir comprises a sealable supply port.

22. The medical device according to claim 18, wherein:
the flow-limiting valve comprises a first valve controlling the opening and closing of the first passage and a second valve controlling the opening and closing of the second passage;
the second valve opens the second passage if the first valve closes the first passage; and
the second valve closes the second passage if the first valve opens the first passage.

23. The medical device according to claim 22, wherein the first valve and the second valve are driven simultaneously by an identical power source.

24. The medical device according to claim 18, wherein:
the driving mechanism comprises a first driving mechanism and a second driving mechanism;
the first driving mechanism controls the flow-limiting valve to open and close the first passage or the second passage; and
the second driving mechanism controls the volume change of the flow feeder to switch the volume of the flow feeder between the first volume and the second volume.

25. The medical device according to claim 24, wherein:
the first driving mechanism and the second driving mechanism are controlled simultaneously;
the first driving mechanism controls the flow-limiting valve to open and close the first passage or the second passage; and
the second driving mechanism simultaneously controls the volume change of the flow feeder to switch the volume of the flow feeder between the first volume and the second volume.

26. The medical device according to claim 24, wherein:
the first driving mechanism and the second driving mechanism are controlled in time delay manner;
the first driving mechanism controls the flow-limiting valve to open and close the first passage or the second passage; and
the second driving mechanism, following the control of the flow-limiting valve by the first driving mechanism, controls the volume change of the flow feeder to switch the volume of the flow feeder between the first volume and the second volume.

27. The medical device according to claim 24, wherein:
when the first driving mechanism keeps the flow-limiting valve in the first state, the flow-limiting valve opens the first passage and closes the second passage, and the second driving mechanism controls the flow feeder to maintain the first volume and receive the predetermined volume of fluid from the reservoir via the first passage; and
when the first driving mechanism keeps the flow-limiting valve in the second state, the flow-limiting valve closes the first passage and opens the second passage, and the second driving mechanism controls the flow feeder to maintain the second volume and provides the predetermined volume of fluid via the second passage.

28. The medical device according to claim 24, wherein:
the flow-limiting valve is of a columnar body and comprises a first through hole and a second through hole;
the first driving mechanism comprises a first driver and a first connector, the first connector being driven by the first driver and connected to the flow-limiting valve;
when the first driver drives the first connector so as to rotate the flow-limiting valve to be in the first state, the first through hole is in communication with the first passage, and the second through hole is not in communication with the second passage; and
when the first driver drives the first connector so as to rotate the flow-limiting valve to be in the second state, the first through hole is not in communication with the first passage, and the second through hole is in communication with the second passage.

29. The medical device according to claim 28, wherein the first through hole comprises a first axis and the second through hole comprises a second axis, the first axis being orthogonal to the second axis.

30. The medical device according to claim 18, wherein:
one side of the reservoir is open and sealed by a first piston; and
another side of the reservoir is in communication with the first passage.

31. The medical device according to claim 30, wherein the first piston is connected to a resilient component, the resilient component being maintained in a compressed state.

32. The medical device according to claim 24, wherein:
the second driving mechanism comprises a second driver and a second connector, the second connector being driven by the second driver and connected to the flow feeder;
when the second driver drives the second connector to change the volume of the flow feeder to maintain the first volume, the fluid flows from the reservoir to the flow feeder via the first passage; and
when the second driver drives the second connector to change the volume of the flow feeder to maintain the second volume, the fluid flows from the flow feeder to the body via the second passage.

33. The medical device according to claim 32, wherein:
one side of the flow feeder is open and sealed by a second piston; and
another side of the flow feeder is in communication with the second passage.

34. The medical device according to claim 33, wherein the second piston is connected to the second connector and driven by the second driver.

35. The medical device according to claim 28, wherein:
the first driver or the second driver is selected from the group consisting of a shape memory alloy, a piezoelectric motor, and a servo motor;
the first connector is selected from the group consisting of a torsion spring and a gear; and
the second connector is selected from the group consisting of a spring and a connecting rod.

36. A medical system, including the medical device of claim 18.

37. The medical system according to claim 36, wherein:
the medical system further comprises an external controller that controls the medical device and a sensing monitor communicatively connected to the external controller;
the sensing monitor obtains data of a physiological parameters of a user and sends the data of the physiological parameters of the user to the external controller; and
the external controller controls the medical device to deliver fluids based on the data of the physiological parameters of the user.
